(19) United States
(12) Patent Application Publication
(10) Pub. No.: US 2015/0209016 A1
(43) Pub. Date: Jul. 30, 2015

(54) REMOTE-CONTROLLED ULTRASOUND DEVICE

(71) Applicant: ADVANCED ECHO TECHNOLOGY, Huisseau-En-Beauce (FR)

(72) Inventors: Eric Lefebvre, Huisseau-En-Beauce (FR); Gwenaël Charron, Naveil (FR)

(21) Appl. No.: 14/417,272

(22) PCT Filed: Jul. 23, 2013
(86) PCT No.: PCT/IB2013/001598

§ 371 Date: Jan. 26, 2015

(30) Foreign Application Priority Data
Jul. 24, 2012 (FR) 12/02093

(51) Int. Cl. A61B 8/00

(52) U.S. Cl. CPC A61B 8/565; A61B 8/4466; A61B 8/4281

(57) ABSTRACT

A remote-controlled ultrasound device including an enclosure envelope containing a tight compartment and a control compartment that are tightly separated by a transverse partition. Sensor means are designed so as to move in the tight compartment, submerged in an ultrasonic-wave-propagating medium, and driving means are arranged in the control compartment for triggering the movement of a support rod at the end of which are mounted the sensor means. The support rod extends through the partition in order to connect the driving means and the sensor means, being mounted in a freely rotational manner inside a tube passing through said partition. The invention is especially applicable to remote ultrasound operations in the medical field.

The present invention relates to a remote-controlled ultrasonic device. This device applies notably to the field of medicine for conducting ultrasonic examinations remotely, a practice known by the name of tele-ultrasonography.

Tele-ultrasonography is an ultrasonic examination performed remotely. In the case of medical tele-ultrasonography which will be described more particularly hereinafter, this remote examination is conducted on a patient at an isolated patient site where no qualified doctor is present. The principle is for a qualified doctor at an expert site to control a real ultrasonic probe contained at the patient site and applied to the patient by an operator who need not have a medical qualification, or by the patient himself. The movements of the hand of the doctor, who controls a joystick that forms a fictitious probe, are transmitted to the real ultrasonic probe present at the end of a robot. The robot reproduces the movements imparted by the doctor more or less in real time and the doctor receives, on a monitor, the image captured by the real probe.

As far as prior art knowledge is concerned mentioned may be made of the work of Professor Philippe Arbeille, who is known to be a specialist in tele-ultrasonography. The development of the ultrasonography device according to the invention makes use of his various works on the subject.

In this context, the invention seeks to propose a remote-controlled ultrasonic device designed to receive sensor-means movement instructions from a control module which calculates the instructions on the basis of the remote movement of a fictitious probe. According to essential features of the invention, the device comprises an outer enclosure inside which there are a sealed compartment and a control compartment which are separated by a transverse partition. The outer enclosure is intended to be pressed against the patient's body and the device is oriented in such a way that it is the sealed compartment that faces the patient. This sealed compartment is filled with a medium through which ultrasonic waves can propagate, for example an oil bath, and the partition ensures that the oil remains confined in this sealed compartment, without overspilling into the control compartment. Sensor means are arranged in the sealed compartment to take images of the patient's body and they are immersed in the medium in order to encourage the propagation of the waves by the sensor means so as to collect the data necessary for creating ultrasonic images. The outer enclosure in the region of the sealed compartment has a wall that is transparent to ultrasonic. The entirety of the sealed compartment may be made from such a material, or alternatively a sighting of appropriate size may simply be created. The sensor means are moved around inside the sealed compartment through the action of drive means which act directly on a support rod on the end of which the sensor means are mounted. The sensor means and the drive means are respectively housed in one of the functional parts which are separated by the partition so that, in order to connect them, the support rod passes through the partition arranged between the control compartment and the sealed compartment. It will be appreciated that the support rod passes through the partition in a sealed manner so as to ensure that the medium with which the sealed compartment is filled does not escape. Advantageously, with this design, the sensor means inside the sealed compartment can be made to move while keeping the outer enclosure stationary.

The device according to the invention thus allows the tele-ultrasonography operation to be performed without there being unwanted movements on the belly of the patient, i.e. without moving the enclosure at its point of contact with the patient's skin. There is no direct contact between the skin and the sensor means, which are the only things that move.

Having an enclosure which completely surrounds the components of the real ultrasonic probe allows for simplified handling on the part of the operator so that these operations can be performed in regions of the body that are not easy to access.

The wave propagating medium in which the sensor means are immersed, which may take the form of an oil bath, makes it possible to ensure correct image acquisition regardless of the position of the mobile sensor means in the stationary enclosure, and the partition makes it possible to ensure that the oil does not overspill out of the sealed compartment as, if it did, it would have the effect both of impeding the movements of the drive means and of allowing an ingress of air that would impede the propagation of waves in the sealed compartment.

According to various advantageous features of the invention, steps are taken to limit the friction on the partition caused by the movement of the support rod that drives the movement of the sensor means on the basis of the movements of the drive means.

On the one hand, the movement of the support rod is controlled in such a way that the position of the point at which the rod passes through the partition is fixed, at the center of this partition. The support rod moves by rocking with respect to this central fixed point and the sensor means arranged at the end of the rod are designed to move in the sealed compartment in a pendular manner with respect to this fixed point arranged at the center of the partition. On the other hand, provision may be made for a tube to be attached to the center of the partition with a seal arranged around the tube and pressed firmly against the partition, and the rod to be passed along inside this tube. The rod is mounted with the freedom to rotate inside the tube and the dimensions of the tube and of the rod are such that the two ends of the rod protrude from the tube, one of them to carry the sensor means and the other to be driven in its own rotation by an actuator. Radial dimensions need to be planned so that the rod can turn within the tube without oil being able to flow between the rod and the tube. Thus, the rod itself may turn without the tube turning. In all cases, it is possible to limit the strain on the partition and prevent it from tearing.

According to one feature of the invention, the tube at its opposite end to the sensor means has a guide ring for the drive means and this guide ring also acts as a support for the actuator engaged directly on the rod to control the rotation of the rod inside the tube. It therefore becomes easier to calculate the instructions for each of the actuators of the drive means and of the rod as these interact directly with the same component.

According to other features of the invention, the sealed compartment has a hemispherical shape with the partition as its base and the sensor means have a radius of curvature equal to the radius of curvature of the sealed compartment, so that the sensor means can move over the surface of the sealed compartment. In this way, through this movement as a surface, it is possible to ensure that the clearance between the sealed compartment and the sensor remains constant, this operating clearance being determined such that the sensor means are as close as possible to the sealed compartment but without contact. The sealed compartment is filled with a medium suited to the propagation of the waves from the sensor means.

It will also be understood that the diameter of the sealed compartment may be as small as the miniaturizing of the sensor permits, while at the same time observing this requirement of equivalent radii of curvature. Therefore, according to the invention, it is possible to offer a remote-controlled ultrasonic device that is more compact, with a control compartment of a width equivalent to that of the sealed compartment. In this way, this device will be easier to apply to tele-ultrasonography equipment carried in space shuttles for example.

The ultrasonic device according to the invention is designed to receive on the one hand, instructions regarding the movement of the sensor means in the sealed compartment which instructions come from a control module associated with the device and, on the other hand, information regarding activation of the piezoelectric transducers that make up the sensor means, these instructions likewise coming from this control module.

Thus, it is possible simultaneously to influence the mechanical movement of the sensor means and the choice of crystals to be activated in order for the sensor means to make whatever observation using an enclosure that remains in a stationary position on the patient's body. The control module needs to be operated using software capable of converting, through calculation, the remote movement data from a fictitious probe into sensor means movement instructions so that the sensor means can gather ultrasonic images similar to those that the qualified doctor would have obtained had he been on the spot.

According to various features that can be found combined in an embodiment set out hereinafter, the drive means comprise two bows arranged at right angles and mounted with a degree of freedom to rotate on a supporting mounting plate on which the enclosure outer is fixed, said support rod being arranged in such a way as to be driven by the rotation of each of the two bows. The partition is also attached to this mounting plate. This then provides an ultrasonic device that is compact because of the mounting plate which fixes both the enclosure outer and the partition. Control of this device is also simplified through the fact that the movement instructions relate only to the control of the two bows and of the associated two actuators, plus the control of any rotation of the rod itself.

The invention also relates to the application of the aforementioned features to a tele-ultrasonography system in which an ultrasonic probe is made to move remotely, the tele-ultrasonography device comprising, on the one hand, equipment on an expert site where a qualified doctor can direct the ultrasonography remotely via an fictitious probe designed to transmit information to a control module in order to direct the movement of the sensor means and, on the other hand, equipment contained at an isolated patient site where the patient who is to be examined is situated, and in which the patient site comprises an ultrasonic device as described hereinabove and designed for performing ultrasonography on of the patient on the basis of the sensor means movement control information which is calculated on the basis of the movement of the fictitious probe at the expert site.

Other features and advantages of the invention will become apparent from the following description of some embodiments, which is illustrated by the following drawings wherein:

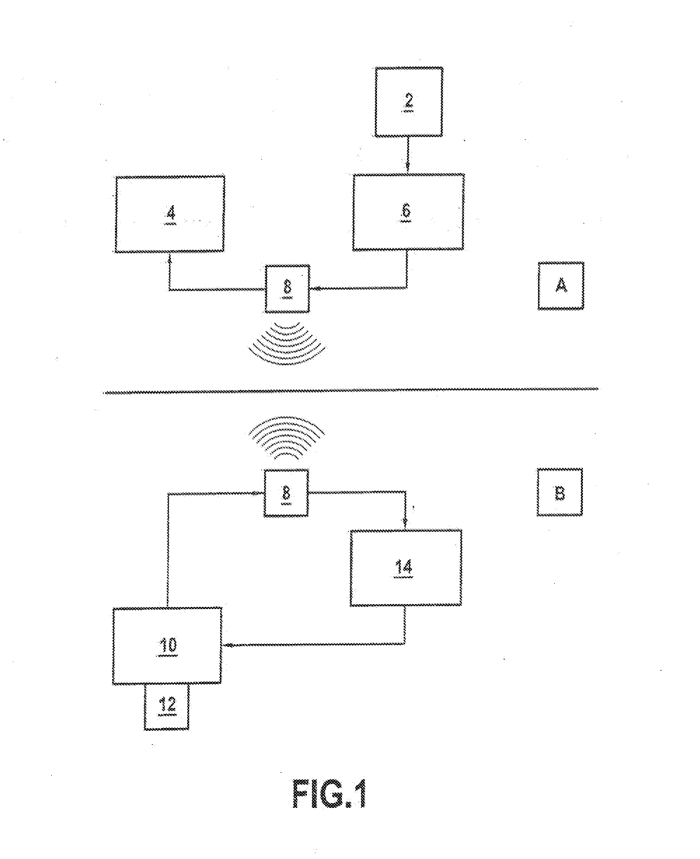

FIG. 1 is a schematic illustration of a principle of tele-ultrasonography which covers the ultrasonic device according to the invention in a particular embodiment;

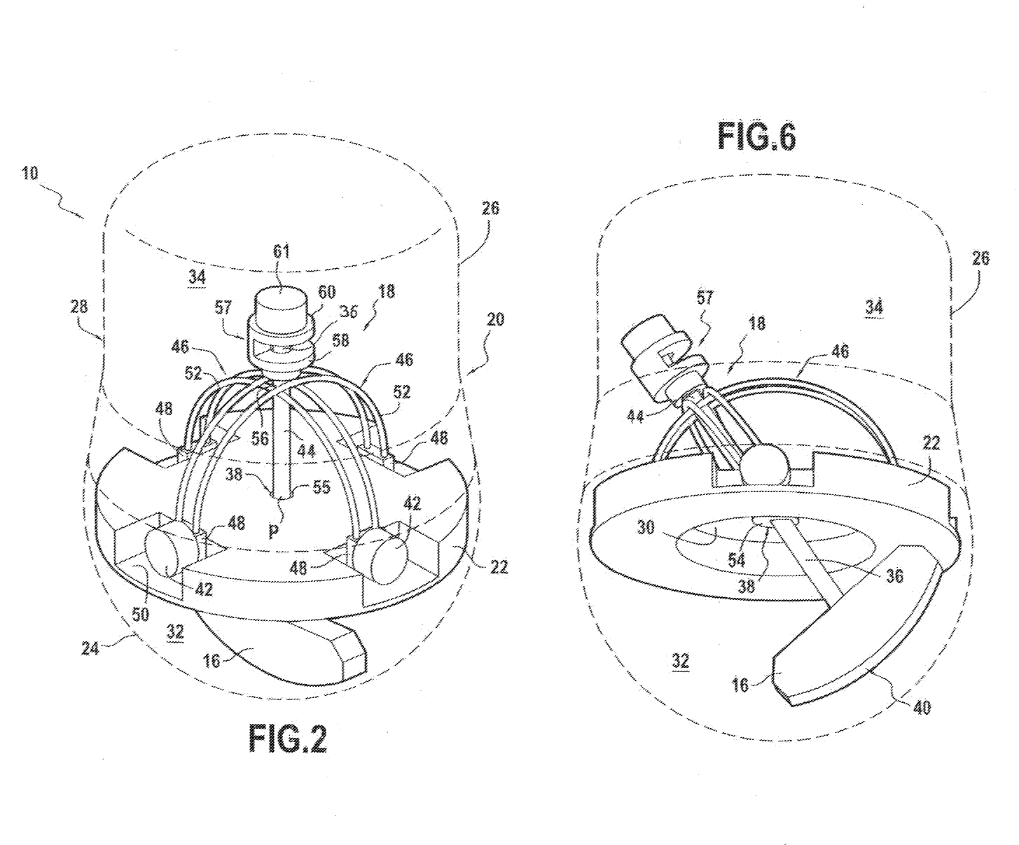

FIG. 2 is a perspective view of an ultrasonic device according to the invention in the context of application to a tele-ultrasonography system illustrated in FIG. 1, the sensor means being depicted in an initial position, in the form of a linear sensor;

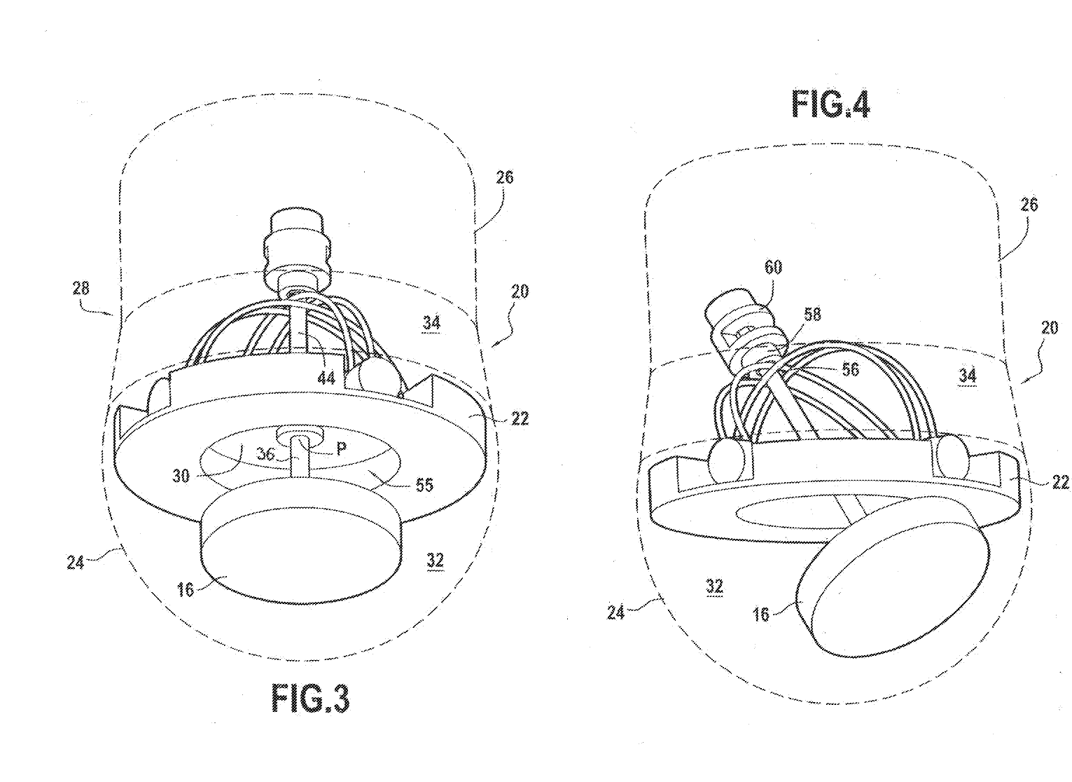

FIG. 3 is a perspective view from beneath of the device illustrated in FIG. 2, the sensor means being depicted in the initial position, in the form of a matrix sensor;

FIG. 4 is a perspective view of the device illustrated in FIG. 3, the sensor means being depicted in a determined first position, in the form of a matrix sensor;

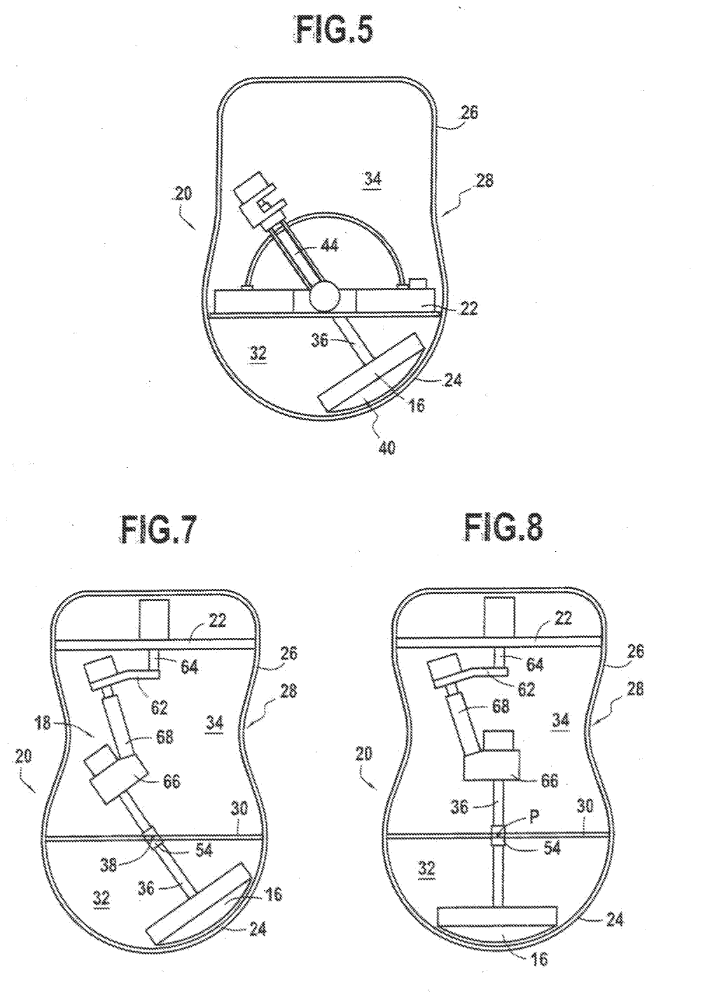

FIG. 5 is a side view of the device of FIG. 4;

FIG. 6 is a perspective view from beneath of the device illustrated in FIG. 2, the sensor means here being depicted in the determined first position, in the form of a linear sensor;

FIGS. 7 and 8 are side views of an ultrasonography device according to the invention in a second particular embodiment, with the sensor means positioned in two distinct positions.

In following, and without in any way restricting the scope thereof, the invention will be considered in the context of the preferred application thereof, unless mentioned otherwise, namely in the case of a tele-ultrasonography system for remote operation by a qualified doctor.

A tele-ultrasonography system is shown on FIG. 1. It comprises, on the one hand, equipment at an expert site A where a qualified doctor can remotely control the echo-cardiography procedure and, on the other hand, equipment embarked at a patient site B where the patient who is to be examined is situated.

The expert site A comprises a fictitious probe 2 that the qualified doctor can handle at will on the basis of the ultrasonic images of the patient, which images are displayed on a monitor 4. The movements of the fictitious probe 2 are measured by sensors provided within the probe and the values measured are transmitted to a processing station 6 wherein these values are encoded as an electronic frame so that they can be transmitted, via transmitters/receivers 8, from the expert site A to the patient site B.

The patient site B contains a remote-controlled echo-cardiography device 10 designed for conducting echo-cardiography tests on a patient by means of a real probe 12, which is controlled by instruction signals calculated on the basis of the movement of the fictitious probe at the expert site A.

The result of the ultrasonic measurements performed by the real probe on the patient is used for producing ultrasonic images which, via the transmitters/receivers, are projected on a monitor 4. The qualified doctor can thus view these images in order to adapt the movement set to the fictitious probe and collect further images.

A control module 14 is designed to control the remote-controlled ultrasonic device so that it reproduces the movements of the fictitious probe 2. For doing so, the control module receives, via the transmitters/receivers 8, encoded values indicative of the movement of the fictitious probe.

As illustrated in FIG. 2, the ultrasonic device 10 comprises sensor means 16 for capturing ultrasonic images and drive means 18 for moving said sensor means, as well as an outer enclosure 20 which surrounds all the equipment and is affixed to a transverse mounting plate 22 that acts also as a support to which the drive means are fixed.

The enclosure comprises two shells, namely a first shell of hemispherical shape 24 and a second shell of cylindrical shape 26, which is deformed to form a means 28 for gripping it. The enclosure is formed, at least in the case of the hemispherical shell, from a material that is transparent to ultrasonic waves.

As may be seen in the first embodiment illustrated in FIG. 2 to 6, the mounting plate also carries a partition 30 which extends transversely to the cylindrical wall of the outer enclosure, substantially in the region where the two shells that form the outer meet, so as to delimit inside the enclosure a sealed compartment 32 and a control compartment 34. The sealed compartment corresponds to the inside of the hemispherical first shell whereas the control compartment corresponds to the cylindrical second shell. It may be seen that the hemispherical shape of the sealed compartment is determined by the partition which forms the base thereof and by the fixed point P at the center of the partition which forms the center of the sphere the lower half of which delimits the sealed compartment.

It may be seen that the sensor means are housed in the sealed compartment whereas the drive means are housed in the control compartment. A support rod 36 is interposed centrally between the sensor means and the drive means and extends notably through the partition center. For that purpose, the partition has a passage 38 through it at its center. The support rod passes through the partition at the center passage so that it allows the sensor means to be set in motion by the prior actuation of the drive means, making sure that the sensor means remain arranged in the sealed compartment whereas the drive means remain in the control compartment.

The sensor means are mounted at the distal end of the support rod. They have a measurement face 40 which is opposite to the support rod and faces toward the surface of the enclosure. This measurement face has a given radius of curvature, slightly smaller than the radius of curvature of the enclosure. In that way, the sensor means are designed to move as a surface along the outer enclosure as soon as they rotate about the same fixed point as the point P defined hereinabove as being the center of the sphere corresponding to the hemispherical shape of the sealed compartment.

Known components necessary for transmitting and receiving ultrasonic waves in order to create ultrasonic images are arranged on the measurement face. Piezoelectric transducers are thus arranged linearly, forming a detection strip (visible in FIGS. 2 and 6) or in two dimensions on the measurement face to form a detection matrix (visible in FIGS. 3 and 4) and these are brought into operation turn by turn about according to the instructions coming from the control module.

A clearance is left between the measurement face of the sensor means and the surface of the outer enclosure so that the sensor means can move around inside the stationary enclosure. This operating clearance, although minimal, may prevent the propagation of waves from and to the sensor means if air circulates between the sensor means and the outer enclosure. For that reason, in the sealed compartment of the remote-controlled ultrasonic device, which extends under the partition and, in this embodiment, under the mounting plate, the sensor means are immersed in a medium through which ultrasonic waves can propagate, in this instance an oil bath. This medium allows the propagation of the ultrasonic waves emitted and received by the sensor means.

The partition is designed to be impervious, in this instance to oil, so that the medium that propagates the waves remains confined to the sealed compartment. Thus the drive means which are wholly housed in the control part are protected from the oil by the partition.

As described previously, the drive means seek to drive the movement of the sensor means via the support rod. The drive means comprise actuators 42 controlled by instructions coming from the control module 14, articulated elements operated by the actuators and a guide tube 44 connected at a first end to the articulated elements and at a second end to the partition, and inside which the support rod is housed.

In the first embodiment illustrated in FIGS. 2 to 6, the articulated elements comprise two bows 46 mounted respectively for pivoting about an axis borne by the mounting plate. These bows are arranged on the mounting plate in such a way that their respective axes of pivoting are mutually perpendicular.

The bows have the overall shape of a semicircle with the diametrically opposite ends each connected to a box 48 which is mounted for rotation on the mounting plate. For each of the bows, one of the boxes is associated, in direct engagement, with a rotary actuator 42 which, according to the instructions coming from the control module, allows the bow to be made to pivot about the pivot axis common to its two boxes. The actuators and the boxes are housed in recesses 50 formed on the transverse mounting plate and which are diametrically opposed in pairs.

The bows are respectively formed of two curved wires 52 which run parallel to one another some distance apart to form a guide way for the guide tube 44.

The inter-axis distance between the wires of one and the same bow is very slightly greater than the diameter of the guide tube so that the latter is in direct contact with the bows. For a given bow, the tube is immobilized laterally by the wires of this bow but can slide axially along the guide way.

The way in which the tube and the bows are arranged is such that the pivoting of one bow causes a movement of the tube which is blocked by the wires of this bow, the tube therefore being made to slide along the guide way of the other bow.

The opposite end of the tube to the drive means is secured to a seal 54, for example a rotary seal, push fitted into the center of the partition in the axial passage so that the pivoting of the bows causes the guide tube to rock about the point at which the tube meets the center of the partition, which so happens to be the fixed point P defined hereinabove. On each of the faces of the mounting plate, as can be seen in FIGS. 2 and 3 for example, bores 55 are provided that have a larger diameter than the outside diameter of the seal, so as to allow the tube to incline with respect to the vertical as it pivots.

In the embodiments illustrated, it may be seen that the tube does not pass through the partition, its end being secured to the partition via the seal. In this way there is created at the center of the partition a tube pivot point which remains stationary such that the tube pivots when the opposite end thereof is driven by the articulated elements. It will be appreciated that a tube that does pass through the partition may be provided, keeping at the center of the partition this fixed point P about which the tube rocks. It is therefore the pivoting of the bows about their respective axis which causes the guide tube to move with respect to the fixed point P, which is situated at the intersection of the perpendicular axes of pivoting of the bows and which is the center of the sphere delimiting the hemispherical shape of the sealed compartment. As will be described hereinafter, simultaneous pivoting of the two bows may be anticipated, or merely the pivoting of one or the other. As illustrated in the figures, the first bow has a diameter at its base that is larger than that of the second bow, and the recesses for housing the boxes and the actuator which are associated with the first bow are more widely separated from one another than those housing the boxes and the actuator which are associated with the second bow. This ensures that one of the bows can pass over the other, but it will be appreciated that this could be achieved using other equivalent means. The essential thing is that the pivoting of each of the two bows is made possible by the fact that a first bow is dimensioned in such a way that it can pass over the top of the other one. A square zone 56 of overlap delimited by four bits of wire parallel in pairs and within which the tube is gripped, pincer-style, is thus created. The pivoting of one bow causes the tube to move with respect to an axis perpendicular to the axis of pivoting and it will be appreciated that the combination of the pivoting movements of the two bows allows the tube to move about many axes.

As illustrated in the figures, the sensor means support rod is housed inside the tube so that it follows the movements of this tube and therefore the pivoting of each of the bows. The sensor means situated at the end of the support rod are thus driven in a movement symmetric to that of the tube with respect to the point of passage. Advantageously, the support rod is mounted with the freedom to rotate inside the tube, so that the support rod can be rotated on itself without the tube turning. As will be indicated hereinafter, this set up offers the advantage of not damaging the partition and the associated seal at the center of which items the tube and the support rod are located.

For that reason, the tube end arranged in the control part is provided with a ring 57 fixed to the tube. The ring extends on the other side of the bows compared with the partition and comprises a base 58, of a diameter greater than the inter-axis distance between the wires of a bow, and a flange 60 to which is attached a third actuator 61 in direct engagement with the support rod housed inside the tube, the third actuator being designed to receive control instructions for setting the rod in rotation about its own axis. The connection between the rod and the third actuator, which is fixed on the ring which extends beyond the bows and of which the base rests on the wires of the upper bow, offers an end stop in terms of translational movement along the axis of the support rod to prevent the latter from escaping and prevent the sensor means from finding themselves pressed firmly against the enclosure without the operating clearance.

The end stop in the opposite direction is provided by the fixing of the tube to the partition and by the fixing of the actuator to the ring secured to the tube. If the device were inverted, the actuator would remain mechanically at the determined distance known to the control module and the support rod in direct engagement with the actuator would not move either.

The control module 14 of the device is designed to provide control signals intended for each of these actuators so as to control the rotational movement of the sensor means in accordance with the rotation instructions on the fictitious probe handled remotely by the qualified doctor. The software built into the control module is parameterized to convert the pivoting about a given point of the fictitious probe into coordinates for the movement of the sensor means and for firing the transducers of these same sensor means. It will be appreciated that the software could be modified to adapt the conversion to suit other drive means or other types of sensor.

Moreover, each actuator is associated with an angular-position sensor, not depicted, which makes it possible to determine the absolute angular position of the bows and of the support rod and therefore the absolute position of the sensor means in the sealed compartment.

It may be seen that, for practical reasons concerned with studying FIGS. 2 to 6, the cables for transmitting the control signals between the actuators, the sensor means and the control module, and the power supply leads, have not been depicted. In the device according to the invention, the presence of these cables and leads does not impact on the sealing of the whole. The cables connecting the sensor means to the control module may extend inside the tube or the rod or may alternatively pass through a cable gland arranged in a sealed manner at the partition, so that in all cases the passage of these cables does not impair the sealing of the partition even though all of the actuators are situated in the control compartment. The cables connecting these actuators may advantageously be led along the mounting plate and up along the enclosure.

It will be appreciated from the foregoing that the sensor means are mounted in such a way as to move in a pendular manner with respect to a determined fixed point so that they perform a surface movement, at a constant distance from the spherical wall of the sealed compartment. According to the invention, this fixed point is located at the center of the partition and that is particularly advantageous in so far as the rotational movements of the support rod and of the sensor means thus do not generate friction and strain on the partition which is not at risk of tearing and can therefore perform its sealing function in a lasting manner. Provision is advantageously made for the support rod to be housed in a tube centered on the partition so as to allow the sensor means themselves to rotate again without this causing friction and strain on the partition.

As a result, the invention is particularly advantageously in that it offers a remote-controlled ultrasonic device for remote operations, and notably for tele-ultrasonography in the field of medicine, in which sensor means are rendered able to move inside an outer enclosure which remains stationary and which covers the entirety of the device applied to the body of the patient. The sensor means which, in the context of use, will be handled by unqualified personnel are protected and the operation is made more comfortable for the patient because the operator does not know how hard he needs to press against the patient's belly and has a tendency to press too hard, thereby nipping the belly as the probe pivots.

The drive means allow the sensor means to be set in motion, this having the effect of limiting the size of these sensor means. It will advantageously be noted that the fact of reducing the size of the sensor means improves the ultrasonic image because the image taking frequency can therefore be increased. Moreover, the fact of combining an electronic scan of the sensor means with the mechanical movements means that the mechanical dimensions of the drive means can be reduced.

As a result, it is possible to adapt the size and radius of curvature of the sensors in order to produce an ultrasonic device that is as compact as possible. Further, having actuators in direct engagement with the articulated elements that are to be actuated contributes toward the desired compactness.

This compactness of the device allows the hemispherical shell to be pressed firmly against the patient's body in regions that are not readily accessible so that ultrasonic images can be taken of regions that were hitherto unfeasible because of the bulkiness of the support of the real probe. Likewise, it has become easy to install the device for use in specific places and, for example, for use in space travel.

The use of the ultrasonic device according to the invention for tele-ultrasonography examinations will now be described. At the patient site, the real probe is positioned in such a way that the hemispherical shell of the outer enclosure is in contact with the working zone, in this instance the body of the patient, facing the region that is to be examined. The ultrasonic images captured by the probe are sent via the transmitters/receivers to the monitor present on the expert site and these images are analyzed by the qualified doctor. To perfect his analysis, the qualified doctor seeks to obtain other images by inclining his fictitious probe, and these movements are reproduced on the patient site via the automated device.

The rotational movements of the fictitious probe are translated and transmitted to the control module via the processing unit and the transmitters/receivers as described hereinabove through the creation of an IT frame based on eight-bit bytes indicative of these movements. The built-in software of the control module then determines by calculation what the movements of the drive means and of the support rod must be in order faithfully to reproduce the movement of the fictitious probe, and corresponding control signals are formulated for each of the actuators of the ultrasonic device.

The software first of all calculates the coordinates on the surface of the enclosure facing which the center of the sensor means is to be positioned and its own rotation angle. Secondly, it calculates, with reference to a previous initial position of the articulated means and of the sensor means, the pivoting of each bow about the respective axis thereof that has to be achieved in order to bring the center of the sensor means to the previously-determined coordinates.

Thus, it will be appreciated that, in order to move from the initial position illustrated in FIG. 3 to the intermediate position illustrated in FIG. 4, the control module has received information coming from the expert site and relating to the inclination of the fictitious probe by the qualified doctor. The built-in software has, on the basis of this information, calculated a control instruction for the actuator associated with the first bow, in this instance the larger-diameter upper bow, a control instruction for the actuator associated with the second bow, and an instruction for the third actuator in direct engagement with the support rod and an instruction to trigger the transducers of the sensor means. In order to arrive here in the intermediate position, the actuator associated with the first bow has been rotated while the actuator associated with the second bow has remained inactive. The tube has moved along the guide path of the second bow parallel to the axis of rotation of this second bow.

It will be appreciated that the software built into the control module, which is designed to convert the movement of the fictitious probe into control signals for the ultrasonic device both for the actuators and for the sensor means, is of a known type and that it can be parameterized differently in order notably to suit particular layouts of the drive means. In this particular instance, the control means receives as input the values pertaining to the bulk of the device installed at the patient site and, notably, the distance between the guide ring borne at one end of the tube and the center of the sensor means, the radius of the bows and the radius of the hemisphere along which the sensor means must move as a surface. Further, the software is designed to calculate the control instructions on the basis of this input data and of the IT frame indicative of the movements of the fictitious probe, which it receives from the processing unit.

In real time, the control module receives, on the one hand, the instructions regarding the movement of the real probe, in order to transmit them in the form of as many control signals as are required in the particular instance, and on the other hand it receives information coming from the actuators and from the angular-position sensors in order in real time to have reliable data regarding the positioning of the drive means so as to be able to calculate the appropriate control signals needed next for directing the whole in accordance with the instructions of the qualified doctor.

The movements performed by the qualified doctor on the fictitious control probe are thus performed remotely by the real probe which reproduces the orientation of the fictitious probe directly on the patient's body and allows ultrasonic images to be captured.

A second embodiment as illustrated in FIGS. 7 and 8 will now be described. The ultrasonic device according to this embodiment differs from that described previously in that the drive means are formed by a set of articulated arms rather than a device involving bows. As a result, the mounting plate is positioned toward the top of the outer enclosure in order to withstand the mechanical strain of the arms and of the actuators. The partition itself extends substantially at the same level as previously, but means for fixing this partition itself with respect to the enclosure need to be provided.

The set of articulated arms comprises an upper arm 62 which rotates as one with a first shaft 64 mounted to pivot on the mounting plate, and it comprises an intermediate arm 66 secured to a second rotation shaft 68 mounted to pivot at the free end of the upper arm. Each shaft is rotationally driven by an associated actuator. The sensor means support rod is mounted at the free end of the intermediate arm. As previously, the support rod is designed to be driven in rotation itself. An arms and shafts arrangement such that the sensor means rotate about a fixed point positioned at the center of this partition is provided. The sensor means support rod passes to the center of the partition and is mounted with the freedom to rotate itself in a tube passing through the center of the partition.

Here again, the control module is designed to provide control signals intended for the actuators respectively associated with each of the elements of the drive means, namely in this instance with each of the articulated arms, so as to control the movement of the sensor means.

Further, as in the first embodiment, each actuator is associated with an angular-position sensor making it possible to determine the absolute angular position of the articulated arms and therefore the position of the sensor means in the sealed compartment.

It will be appreciated that the invention is not restricted only to the devices according to the embodiments explicitly described with reference to FIGS. 1 to 8, and it should also be noted that the invention is not restricted to the preferred embodiment relating to tele-ultrasonography operations in the field of medicine.

1. A tele-ultrasonography device comprising an outer enclosure inside which there are a sealed compartment, in which sensor means are designed to move, these being immersed in a medium through which ultrasonic waves can propagate and a control compartment which is separated in a sealed manner from said sealed compartment by a transverse partition, drive means being housed in the control compartment for setting in motion a support rod at the end of which the sensor means are mounted, wherein said sensor means support rod is mounted free to rotate inside a tube passing through said partition, at a fixed point located at the center of the partition so that the sensor means are able to move in said sealed compartment in a pendular manner with respect to this fixed point, a seal arranged around the tube being pressed firmly against the partition.

2. The device as claimed in claim 1, wherein the tube, at its opposite end to the sensor means, bears a ring which extends beyond the drive means and which bears an actuator in direct engagement on the support rod mounted free to rotate inside the tube.

3. The device as claimed in claim 1, wherein the sealed compartment has a hemispherical shape the base of which is formed by said partition and the center of which is said fixed point at the center of said partition, and in that the sensor means have a radius of curvature slightly smaller than the radius of curvature of the sealed compartment, so as to ensure that the sensor means move as a surface over the surface of the sealed compartment.

4. The device as claimed in claim 1, wherein it is associated with a control module and that it is able to receive, from said control module, instructions for operating the drive means in the control compartment for the movement of the sensor means in the sealed compartment and information regarding the activation of the piezoelectric elements that the sensor means include.

5. The device as claimed in claim 1, wherein the means comprise two bows arranged at right angles and mounted with pivoting on a mounting plate on which the outer enclosure is fixed, said support rod being arranged in such a way as to be driven by the pivoting of each of the two bows.

6. The device as claimed in claim 5, wherein the partition is attached to said mounting plate.

7. The device as claimed in claim 1, wherein the drive means are formed by a set of articulated arms which comprises an upper arm that rotates as one with a first shaft pivot-mounted on said mounting plate, an intermediate arm secured to a second rotation shaft pivot-mounted at the free end of the upper arm, said sensor means support rod being mounted at the free end of the intermediate arm, said arms and said shafts being arranged in such a way that the sensor means at the end of the support rod move in a pendular manner about a fixed point arranged at the center of said partition attached to the outer enclosure some distance away from a mounting plate on which the outer enclosure and the drive means are fixed.

8. A tele-ultrasonography assembly in which an ultrasonic probe is moved remotely and which comprises, on the one hand, equipment on an expert site where a qualified doctor can direct the ultrasonography remotely and, on the other hand, equipment contained at a patient site where the patient who is to be examined is situated, wherein the patient site comprises a tele-ultrasonography device as claimed in claim 1, designed for performing ultrasonography on the patient on the basis of the control information stemming from the movement of an fictitious probe at the expert site.
